(19) United States
(12) Patent Application Publication
(10) Pub. No.: US 2011/0177523 A1
(43) Pub. Date: Jul. 21, 2011

(54) METHODS OF ANALYZING SAMPLES FOR BACTERIA USING WHOLE CELL CAPTURE AND ATP ANALYSIS

(76) Inventors: G. Marco Bommarito, Stillwater, MN (US); Joseph J. Stoffel, Hastings, MN (US); Sridhar V. Dasaratha, Bangalore (IN); Jason W. Bjork, Newport, MN (US); Patrick A. Mach, Shorewood, MN (US); Mara S. Reif-Wenner, Red Wing, MN (US); Chunmet Guo, Woodbury, MN (US); Heather M. Webb, Woodbury, MN (US)

(21) Appl. No.: 12/867,317

(22) PCT Filed: Feb. 19, 2009
(86) PCT No.: PCT/US2009/034450

§ 371 Date: Mar. 31, 2011

Related U.S. Application Data
(60) Provisional application No. 61/066,336, filed on Feb. 20, 2008.

(51) Int. Cl. G01N 33/53

(52) U.S. Cl. 435/7.1

(57) ABSTRACT

The invention relates to methods of capturing bacterial whole cells that includes the use of one or more antibodies having antigenic specificities for one or more distinct analytes characteristic of the specific bacterium, followed by analyzing the target whole cells using a direct or indirect ATP assay.

### CROSS REFERENCE TO RELATED APPLICATIONS

This application claims the benefit of U.S. Provisional Application Ser. No. 61/066,336, filed Feb. 20, 2008, which is incorporated herein by reference.

### BACKGROUND

The emergence of bacteria having resistance to commonly used antibiotics is an increasing problem with serious implications for the treatment of infected individuals. Accordingly, it is of increasing importance to determine the presence of such bacteria at an early stage and in a relatively rapid manner to gain better control over such bacteria. This also applies to a variety of other microbes.

One such microbe of significant interest is Staphylococcus aureus (“S. aureus”). This is a pathogen causing a wide spectrum of infections including: superficial lesions such as small skin abscesses and wound infections; systemic and life threatening conditions such as endocarditis, pneumonia and septicemia; as well as toxinoses such as food poisoning and toxic shock syndrome. Some strains (e.g., Methicillin-Resistant S. aureus) are resistant to all but a few select antibiotics.

Current techniques for the detection of microbes, particularly bacteria resistant to antibiotics, are generally time consuming and typically involve culturing the bacteria in pure form. One such technique for the identification of pathogenic staphylococci associated with acute infection, i.e., S. aureus in humans and animals and S. intermedius and S. hyicus in animals, is based on the microbe's ability to clot plasma. At least two different coagulase tests have been described: a tube test for free coagulase and a slide test for “cell bound coagulase” or clumping factor. The tube coagulase test typically involves mixing an overnight culture in brain heart infusion broth with reconstituted plasma, incubating the mixture for 4 hours and observing the tube for clot formation by slowly tilting the tube for clot formation. Incubation of the test overnight has been recommended for S. aureus since a small number of strains may require longer than 4 hours for clot formation. The slide coagulase test is typically faster and more economical; however, 10% to 15% of S. aureus strains may yield a negative result, which requires that the isolate be reexamined by the tube test.

Although methods of detecting S. aureus, as well as other microbes, have been described in the art, there would be advantage in improved methods of detection.

### SUMMARY

In certain embodiments, the invention provides methods for capturing whole bacterial cells followed by analysis of adenosine triphosphate (ATP) either directly or indirectly. The methods involve the use of one or more antibodies having antigenic specificities for one or more distinct analytes characteristic of the specific bacterium (preferably, two or more antibodies having antigenic specificities for two or more distinct analytes characteristic of the specific bacterium). If more than one antibody is used, the two or more antibodies are preferably cooperative in their binding characteristics. That is, they are capable of simultaneously binding to distinct regions of the target analyte(s) or optimally are found to be of complementary binding whereby the binding of a distinct analyte is enhanced by the binding of another antibody.

In one such embodiment, the present invention provides methods of analyzing for a specific bacterium, wherein the methods include: providing a sample suspected of including target whole cells comprising one or more analytes characteristic of a specific bacterium; providing one or more antibodies having antigenic specificities for one or more distinct analytes characteristic of the specific bacterium, wherein the antibodies are selected from the group consisting of MAb-76, MAb-107, affinity-purified

RxClf40, affinity-purified GxClf40, MAb 12-9, fragments thereof, and combinations thereof; providing a solid support material comprising magnetic particles; providing contact between the sample, the solid support material, and the one or more antibodies under conditions effective to capture target whole cells with one or more analytes characteristic of a specific bacterium, if present; separating the captured target whole cells from the sample; lysing the target whole cells to form a lysate; and analyzing for the presence or absence of the specific bacterium by analyzing the lysate for ATP directly or indirectly.

In one preferred embodiment, the one or more (preferably, two or more) antibodies are attached to the solid support material forming an analyte-binding material, and the method includes providing contact between the sample and the analyte-binding material under conditions effective to capture whole cells with one or more analytes characteristic of a specific bacterium, if present. Providing contact between the sample and the analyte-binding material can include simultaneous and/or sequential, preferably simultaneous, contact between the sample and the one or more antibodies.

In another preferred embodiment, providing contact between the sample, the solid support material, and the one or more antibodies includes providing contact between the one or more antibodies and the sample to form antibody-bound whole cells, and subsequently providing contact between the antibody-bound whole cells and the solid support material.

In certain embodiments, the specific bacterium comprises a Gram positive bacterium, particularly Staphylococcus aureus.

In certain embodiments, each particle has at least two antibodies that bind different analytes disposed thereon.

In certain embodiments, the target whole cells are removed from the magnetic particles prior to lysing.

In certain embodiments, analyzing for the presence or absence of the specific bacterium by analyzing for ATP directly or indirectly includes: contacting the lysate with a solution containing adenosine diphosphate (ADP) under conditions effective to produce ATP by any adenylate kinase present; and detecting for the presence or absence of produced ATP. In certain embodiments, detecting for the presence or absence of produced ATP comprises measuring the amount of ATP produced and relating that to the presence and/or amount of specific bacterium or intracellular material characteristic of the specific bacterium. In certain embodiments, detecting for the presence or absence of produced ATP comprises contacting the mixture containing the lysate and ADP with a luciferase/luciferin reagent to produce light proportional to the amount of ATP produced, and detecting the light with a luminometer.

In certain embodiments, analyzing for the presence or absence of the specific bacterium by analyzing for ATP directly or indirectly includes: contacting the lysate with a luciferase/luciferin reagent to produce light proportional to the amount of ATP present; and detecting the light using a luminometer. In certain embodiments, analyzing for the presence or absence of the specific bacterium further includes measuring the amount of ATP present and relating that to the amount of specific bacterium or intracellular material characteristic of the specific bacterium present.

The present invention also provides a method of analyzing a sample for a bacterium, the method includes: providing a sample suspected of including target whole cells comprising one or more analytes characteristic of a specific bacterium; providing a solid support material comprising magnetic particles having attached thereto one or more antibodies having antigenic specificities for one or more distinct analytes characteristic of the specific bacterium, wherein the antibodies are selected from the group consisting of MAb-76, MAb-107, affinity-purified RxClf40, affinity-purified GxClf40, MAb 12-9, fragments thereof, and combinations thereof; providing contact between the sample and the magnetic particles having the one or more antibodies attached thereto under conditions effective to capture target whole cells with one or more analytes characteristic of a specific bacterium, if present; separating the captured target whole cells from the sample; lysing the target whole cells to form a lysate and release adenosine triphosphate (ATP) if present; contacting the lysate with a luciferase/luciferin reagent to produce light proportional to the amount of ATP present; detecting the light using a luminometer; and measuring the amount of ATP present and relating that to the amount of the specific bacterium or intracellular material present characteristic of the specific bacterium.

The present invention further provides a method of analyzing a sample for a bacterium, the method includes: providing a sample suspected of including target whole cells comprising one or more analytes characteristic of a specific bacterium;

providing a solid support material comprising magnetic particles having attached thereto one or more antibodies having antigenic specificities for one or more distinct analytes characteristic of the specific bacterium, wherein the antibodies are selected from the group consisting of MAb-76, MAb-107, affinity-purified RxClf40, affinity-purified GxClf40, MAb 12-9, fragments thereof, and combinations thereof; providing contact between the sample and the magnetic particles having the one or more antibodies attached thereto under conditions effective to capture target whole cells with one or more analytes characteristic of a specific bacterium, if present; separating the captured target whole cells from the sample; lysing the target whole cells to form a lysate; contacting the lysate with a solution containing adenosine diphosphate (ADP) under conditions effective to produce adenosine triphosphate (ATP) by any adenylate kinase present; and measuring the amount of adenosine triphosphate produced and relating that to the amount of the specific bacterium or intracellular material characteristic of the specific bacterium.

### DEFINITIONS

“Whole cell” means a biologically active bacterial cell that retains its structure during separation from other biological materials, but does not necessarily need to be able to reproduce.

The terms “analyte” and “antigen” are used interchangeably and refer to various molecules (e.g., Protein A) or epitopes of molecules (e.g., different binding sites of Protein A), or whole cells of the microorganism, that are characteristic of a microorganism (i.e., microbe) of interest. These include components of cell walls (e.g., cell-wall proteins such as protein A, and Clumping Factor, which is a cell wall-associated fibrinogen receptor that is found in S. aureus), external cell components (e.g., capsular polysaccharides and cell-wall carbohydrates), etc.

“Magnetic particles” means particles or particle agglomerates comprised of ferromagnetic, paramagnetic, or superparamagnetic particles, including dispersions of said particles in a polymer bead.

The words “preferred” and “preferably” refer to embodiments of the invention that may afford certain benefits, under certain circumstances. However, other embodiments may also be preferred, under the same or other circumstances.

Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

As used herein, “a,” “an,” “the,” “at least one,” and “one or more” are used interchangeably. Thus, for example, an analyte-binding material that comprises “an” antibody can be interpreted to mean that the analyte-binding material includes “one or more” antibodies that bind different analytes.

The term “and/or” means one or all of the listed elements or a combination of any two or more of the listed elements.

Also herein, the recitations of numerical ranges by endpoints include all numbers subsumed within that range (e.g., 1 to 5 includes 1, 1.5, 2, 2.75, 3, 3.80, 4, 5, etc.).

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### DETAILED DESCRIPTION OF ILLUSTRATIVE EMBODIMENTS

The present invention involves various methods of capturing whole cells of a bacterium of interest from a sample based on the use of one or more analytes characteristic of the bacterium of interest. Specifically, the capture methods of the present invention include the use of one or more antibodies (preferably two or more) having antigenic specificities for one or more distinct analytes (preferably two or more) characteristic of the specific bacterium. If two or more antibodies are used, they are preferably cooperative in their binding characteristics. That is, they are capable of simultaneously binding to distinct regions of the target analyte(s) or optimally are found to be of complementary binding whereby the binding of a distinct analyte is enhanced by the binding of another antibody.

Once attached to the magnetic particles, the target whole cells can be removed from the sample prior to further analysis. The advantage of selectively binding target whole cells prior to analysis and separating them from the remainder of the sample is that it selectively concentrates the cells and can provide better sensitivity and specificity. It also eliminates the inhibitors that may be present in the complex sample.

Techniques of analyzing for bacteria in methods of the present invention involve the analysis of adenosine triphosphate (ATP) either directly or indirectly. Prior to such analysis, the captured (target) whole cells can be lysed without being released from the magnetic particles or after release therefrom.

The present invention is advantageous in many situations where whole cell capture is part of the sample preparation step prior to detection or further analysis. It is known that the expression of a target protein can vary significantly for a given strain of bacteria. In certain situations, capture of strains that are well captured with a single antibody technique can be used. In other situations, a single antibody against a single antigen of the targeted bacteria can result in some strains of the bacteria showing poor capture efficiency or not being captured at all. For these strains the sample preparation step would result in highly reduced availability of the bacteria for detection. As a result, this will increase the number of false negatives for the detection technique and this also has an adverse effect on the detection limit of the assay. By having a mix of particles coated with different antibodies or having different antibodies coated on the same particle, it is possible to increase the capture efficiency of bacterial strains showing poor or no capture with a single antibody. Thus, the assay sensitivity as well as the detection limit of an assay using whole cell capture can be improved by using a preferred method of this invention with two or more antibodies having antigenic specificities for two or more distinct analytes characteristic of the specific bacterium.

Preferably, relatively small volumes of test sample can be used. Although test sample volumes significantly greater than 2 milliliters (mL) may be utilized, test samples on the order of 500 microliters (μL) are typically sufficient for methods of the present invention, although smaller sample sizes may be possible.

Preferably, using methods of the present invention, the capture time can be relatively short. For example, the capture time can be less than 30 minutes, less than 15 minutes, less than 5 minutes, less than 60 seconds, and even as short as 30 seconds.

Bacteria of particular interest include Gram positive and Gram negative bacteria. Particularly relevant organisms include members of the family Enterobacteriaceae, or the family Micrococcaceae or the genera Staphylococcus spp., Streptococcus spp., Pseudomonas spp., Enterococcus spp., Salmonella spp., Legionella spp., Shigella spp. Yersinia spp., Enterobacter spp., Escherichia spp., Bacillus spp., Listeria spp., Vibrio spp., Corynebacteria spp.as well as herpes virus, Aspergillus spp., Fusarium spp., and Candida spp. Particularly virulent organisms include Staphylococcus aureus (including resistant strains such as Methicillin Resistant Staphylococcus aureus (MRSA)), S. epidermidis, Streptococcus pneumoniae, S. agalactiae, S. pyogenes, Enterococcus faecalis, Vancomycin Resistant Enterococcus (VRE), Vancomycin Resistant Staphylococcus aureus (VRSA), Vancomycin Intermediate-resistant Staphylococcus aureus (VISA), Bacillus anthracis, Pseudomonas aeruginosa, Escherichia coli, Aspergillus niger, A. fumigatus, A. clavatus, Fusarium solani, F. oxysporum, F. chlamydosporum, Listeria monocytogenes, Listeria ivanovii, Vibrio cholera, V. parahemolyticus, Salmonella cholerasuis, S. typhi, S. typhimurium, Candida albicans, C. glabrata, C. krusei, Enterobacter sakazakii, E. coli 0157 and multiple drug resistant Gram negative rods (MDR).

Of particular interest are Gram positive bacteria, such as Staphylococcus aureus. Typically, these can be detected by detecting the presence of a cell-wall component characteristic of the bacteria, such as a cell-wall protein. Also, of particular interest are antibiotic resistant microbes including MRSA, VRSA, VISA, VRE, and MDR. Typically, these can be detected by additionally detecting the presence of an internal cell component, such as a membrane protein, transport protein, enzyme, etc., responsible for antibiotic resistance.

Preferred methods of the present invention could be used to capture whole bacterial cells from a sample using separate molecules (e.g., molecules like protein A and Clumping Factor for analysis of Staphylococcus aureus) or two different epitopes of the same molecule (e.g., a protein). Such analytes include, for example, cell-wall proteins such as protein A and microbial surface components recognizing adhesive matrix molecules (MSCRAMMs) such as fibrinogen-binding proteins (e.g., clumping factors), fibronectin-binding proteins, collagen-binding proteins, heparin-related polysaccharides binding proteins, and the like. Protein A and clumping factors, such as fibrinogen-binding factors and clumping factors A, B, and Efb, are also particularly useful in methods of detecting the presence of Staphylococcus aureus. Other cell-wall components of interest include capsular polysaccharides and cell-wall carbohydrates (e.g., teichoic acid and lipoteichoic acid).

Species of interest can be analyzed in a test sample that may be derived from a wide variety of sources, such as a physiological fluid, e.g., blood, saliva, ocular lens fluid, synovial fluid, cerebral spinal fluid, pus, sweat, exudate, urine, mucus, mucosal tissue (e.g., buccal, gingival, nasal, ocular, tracheal, bronchial, gastrointestinal, rectal, urethral, ureteral, vaginal, cervical, and uterine mucosal membranes), lactation milk, feces, or the like. Further, the test sample may be derived from a body site, e.g., wound, skin, anterior nares, nasopharyngeal cavity, nasal cavities, anterior nasal vestibule, scalp, nails, outer ear, middle ear, mouth, rectum, vagina, axilla, perineum, anus, rectum, or other similar site.

Besides physiological fluids, other test samples may include other liquids as well as solid(s) dissolved in a liquid medium. Samples of interest may include process streams, water, soil, plants or other vegetation, air, surfaces (e.g., contaminated), and the like.

The art describes various patient sampling techniques for the detection of bacteria, such as S. aureus. Such sampling techniques are suitable for the methods of the present invention as well. For example, it is common to obtain a sample from wiping the nares of a patient, e.g., patient's anterior nares, by swabbing with a sterile swab or sampling device. For example, one swab is used to sample each subject, i.e., one swab for both nares. The sampling can be performed, for example, by inserting the swab dry or pre-moistened with an appropriate solution into the anterior tip of the subject's nares and rotating the swab for two complete revolutions along the nares' mucosal surface.

A wide variety of swabs or other sample collection devices are commercially available, for example, from Puritan Medical Products Co. LLC, Guilford, Me., under the trade designation PURE-WRAPS, or from Copan Diagnostics, Inc., Murrietta, Calif., under the trade designations microRheologics nylon flocked swab and ESwab Collection and Transport System. A sample collection means such as that disclosed, for example, in U.S. Pat. No. 5,879,635 (Nason) can also be used if desired. Swabs can be of a variety of materials including cotton, rayon, calcium alginate, Dacron, polyester, nylon, polyurethane, and the like.

The sample collection device (e.g., swab) can then be cultured directly, analyzed directly, or extracted (e.g., by washing, elution by vortexing) with an appropriate solution. Such extraction (i.e., elution) solutions typically include water and can optionally include a buffer and at least one surfactant. An example of an elution buffer includes, for example, phosphate buffered saline (PBS), which can be used in combination, for example, with TWEEN 20 (polyoxyethylene sorbitan monolaurate, available from Sigma-Aldrich Corp.) or PLURONIC L64 (poly(oxyethylene-co-oxypropylene) block copolymer, available from BASF Corp.). Other extraction solutions function to maintain specimen stability during transport from sample collection site to sample analysis sites. Examples of these types of extraction solutions include Amies' and Stuart's transport media.

The test sample (e.g., liquid) may be subjected to treatment prior to further analysis. This includes concentration, precipitation, filtration, centrifugation, distillation, dialysis, dilution, inactivation of natural components, addition of reagents, chemical treatment, etc.

The sample is contacted with appropriate antibodies, which can be attached to magnetic particulate material. Bound cells may be eluted from the support to obtain purified target analytes or processed while attached to the solid support material.

One or more (preferably, two or more) antibodies, such as an S. aureus antibody, are employed as an S. aureus reactant. “S. aureus antibody” refers to an immunoglobulin having the capacity to specifically bind a given antigen inclusive of antigen binding fragments thereof S. aureus antibodies are commercially available from Sigma-Aldrich and Accurate Chemical. Further, other S. aureus antibodies, such as the monoclonal antibody Mab 12-9, are described in U.S. Pat. No. 6,979,446. In certain preferred embodiments, an antibody is selected from those described herein (e.g., selected from the group consisting of MAb-76, MAb-107, affinity-purified RxClf40, affinity-purified GxClf40, MAb 12-9), fragments thereof, and combinations thereof. Such antibodies are also disclosed in U.S. Patent Application Publication No. 2008-0118937-A1 and PCT Publication No. WO 2008/140570, both entitled “ANTIBODY WITH PROTEIN A SELECTIVITY,” and in U.S. patent application Ser. No. 11/562,747, filed on Nov. 22, 2006, and PCT Publication No. WO 2008/143697, both entitled “ANTIBODY WITH PROTEIN A SELECTIVITY,” and in U.S. Patent Application Ser. No. 60/867,089, filed on Nov. 22, 2006 and U.S. patent application Ser. No. 11/943,168, filed on Nov. 20, 2007, both of which are entitled “SPECIFIC ANTIBODY SELECTION BY SELECTIVE ELUTION CONDITIONS.”

Preferred antibodies are monoclonal antibodies. Particularly preferred are monoclonal antibodies that bind to Protein A of Staphylococcus aureus (also referred to herein as “S. aureus” or “Staph A”). Particularly preferred antibodies are MAb-76, MAb-107, fragments thereof, and combinations thereof.

More particularly, in one embodiment, suitable monoclonal antibodies, and antigen binding fragments thereof, are those that demonstrate immunological binding characteristics of monoclonal antibody 76 as produced by hybridoma cell line 358A76.1. Murine monoclonal antibody 76 is a murine IgG2A, kappa antibody isolated from a mouse immunized with Protein A. In accordance with the Budapest Treaty, hybridoma 358A76.1, which produces monoclonal antibody 76, was deposited on Oct. 18, 2006 in the American Type Culture Collection (ATCC) Depository, 10801 University Boulevard, Manassas, Va. 20110-2209, and was given Patent Deposit Designation PTA-7938 (also referred to herein as accession number PTA-7938). The hybridoma 358A76.1 produces an antibody referred to herein as “Mab 76.” Mab 76 is also referred to herein as “Mab76,” “Mab-76,” “MAb-76,” “monoclonal 76,” “monoclonal antibody 76,” “76,” “M76,” or “M 76,” and all are used interchangeably herein to refer to immunoglobulin produced by hybridoma cell line 358A76.1 as deposited with the American Type Culture Collection (ATCC) on Oct. 18, 2006, and assigned Accession No. PTA-7938.

In another embodiment, suitable monoclonal antibodies, and antigen binding fragments thereof, are those that demonstrate immunological binding characteristics of monoclonal antibody 107 as produced by hybridoma cell line 358A107.2. Murine monoclonal antibody 107 is a murine IgG2A, kappa antibody isolated from a mouse immunized with Protein A. In accordance with the Budapest Treaty, hybridoma 358A107.2, which produces monoclonal antibody 107, was deposited on October 18, 2006 in the American Type Culture Collection (ATCC) Depository, 10801 University Boulevard, Manassas, Va. 20110-2209, and was given Patent Deposit Designation PTA-7937 (also referred to herein as accession number PTA-7937). The hybridoma 358A107.2 produces an antibody referred to herein as “Mab 107.” Mab 107 is also referred to herein as “Mab107,” “Mab-107,” “MAb-107,” “monoclonal 107,” “monoclonal antibody 107,” “107,” “M107,” or “M 107,” and all are used interchangeably herein to refer to immunoglobulin produced by the hybridoma cell line as deposited with the American Type Culture Collection (ATCC) on Oct. 18, 2006, and given Accession No. PTA-7937.

Suitable monoclonal antibodies are also those that inhibit the binding of monoclonal antibody MAb-76 to Protein A of S. aureus. The present invention can utilize monoclonal antibodies that bind to the same epitope of Protein A of S. aureus that is recognized by monoclonal antibody MAb-76. Methods for determining if a monoclonal antibody inhibits the binding of monoclonal antibody MAb-76 to Protein A of S. aureus and determining if a monoclonal antibody binds to the same epitope of Protein A of S. aureus that is recognized by monoclonal antibody MAb-76 are well known to those skilled in the art of immunology.

Suitable monoclonal antibodies are also those that inhibit the binding of monoclonal antibody MAb-107 to Protein A of S. aureus. The present invention can utilize monoclonal antibodies that bind to the same epitope of Protein A of S. aureus that is recognized by monoclonal antibody MAb-107. Methods for determining if a monoclonal antibody inhibits the binding of monoclonal antibody MAb-107 to Protein A of S. aureus and determining if a monoclonal antibody binds to the same epitope of Protein A of S. aureus that is recognized by monoclonal antibody MAb-107 are well known to those skilled in the art of immunology.

Suitable monoclonal antibodies are those produced by progeny or derivatives of this hybridoma and monoclonal antibodies produced by equivalent or similar hybridomas.

Also included in the present invention include various antibody fragments, also referred to as antigen binding fragments, which include only a portion of an intact antibody, generally including an antigen binding site of the intact antibody and thus retaining the ability to bind antigen. Examples of antibody fragments include, for example, Fab, Fab′, Fd, Fd′, Fv, dAB, and F(ab′)₂ fragments produced by proteolytic digestion and/or reducing disulfide bridges and fragments produced from an Fab expression library. Such antibody fragments can be generated by techniques well known in the art.

Monoclonal antibodies useful in the present invention include, but are not limited to, humanized antibodies, chimeric antibodies, single chain antibodies, single-chain Fvs (scFv), disulfide-linked Fvs (sdFv), Fab fragments, F(ab') fragments, F(ab')₂ fragments, Fv fragments, diabodies, linear antibody fragments produced by a Fab expression library, fragments including either a VL or VH domain, intracellularly-made antibodies (i.e., intrabodies), and antigen-binding antibody fragments thereof.

Monoclonal antibodies useful in the present invention may be of a wide variety of isotypes. The monoclonal antibodies useful in the present invention may be, for example, murine IgM, IgG1, IgG2a, IgG2b, IgG3, IgA, IgD, or IgE. The monoclonal antibodies useful in the present invention may be, for example, human IgM, IgG1, IgG2, IgG3, IgG4, IgAl, IgA2, IgD, or IgE. In some embodiments, the monoclonal antibody may be murine IgG2a, IgG1, or IgG3. With the present invention, a given heavy chain may be paired with a light chain of either the kappa or the lambda form.

Monoclonal antibodies useful in the present invention can be produced by an animal (including, but not limited to, human, mouse, rat, rabbit, hamster, goat, horse, chicken, or turkey), chemically synthesized, or recombinantly expressed. Monoclonal antibodies useful in the present invention can be purified by a wide variety of methods known in the art for purification of an immunoglobulin molecule, for example, by chromatography (e.g., ion exchange, affinity, and sizing column chromatography), centrifugation, differential solubility, or by a wide variety of other standard techniques for the purification of proteins.

Suitable antibodies also include a high avidity anti-Staphylococcus aureus clumping factor protein polyclonal antibody preparation that detects recombinant clumping factor (rClf40) protein of S. aureus at a concentration of preferably at least 1 picogram per milliliter (pg/mL), and more preferably up to 100 pg/mL. Suitable antibodies also include a high avidity anti-Staphylococcus aureus clumping factor protein polyclonal antibody preparation demonstrating at least a 4-fold increase in detection sensitivity in comparison to a Staphylococcus aureus clumping factor protein antiserum.

In certain embodiments, a high avidity anti-Staphylococcus aureus clumping factor protein polyclonal antibody preparation is useful, wherein the high avidity anti-S. aureus clumping factor protein polyclonal antibody preparation is prepared by a method that includes obtaining antiserum from an animal immunized with recombinant clumping factor (rClf40) protein of S. aureus; binding the antiserum to an S. aureus clumping factor (Clf40) protein affinity column; washing the column with a wash buffer having 0.5 M salt and a pH of 4; and eluting the high avidity anti-S. aureus clumping factor protein polyclonal antibody preparation from the column with an elution buffer with a pH of 2. Herein, the high avidity anti-Staphylococcus aureus clumping factor polyclonal antibody preparations from rabbits and goats are referred to as affinity-purified RxClf40 and affinity-purified GxClf40, respectively. In some embodiments, the high avidity anti-Staphylococcus aureus clumping factor protein polyclonal antibody preparation may be obtained by a method that further includes enriching the antiserum for the IgG class of antibodies prior to binding the antiserum to an S. aureus clumping factor (C1f40) protein affinity column. Such enrichment may eliminate non-immunoglobulin proteins from the preparation and/or enrich for the IgG class of antibodies within the sample.

As used herein, antiserum refers to the blood from an immunized host animal from which the clotting proteins and red blood cells (RBCs) have been removed. An antiserum to a target antigen may be obtained by immunizing a wide variety of host animals. A wide variety of immunization protocols may be used.

Antibody avidity is a measure of the functional affinity of a preparation of polyclonal antibodies. Avidity is the compound affinity of multiple antibody/antigen interactions. That is, avidity is the apparent affinity of antigen/antibody binding, not the true affinity. Despite the heterogeneity of affinities in most antisera, one can characterize such populations by defining an average affinity (K₀).

The analyte-binding material includes magnetic particulate materials such as ferromagnetic, paramagnetic, and superparamagnetic materials. Preferably, such magnetic particles (e.g., beads) have an average particle size (i.e., the longest dimension of an individual particle, e.g., diameter) of less than 2 microns, and preferably, within a range of 0.05 micron to 1 micron. For example, magnetic particles functionalized with various groups such as carboxyl, amine, and tosyl are commercially available from various commercial sources such as Invitrogen (Carlsbad, Calif.) and Ademtech (Pessac, France). Streptavidin coated particles are also available from several sources such as Invitrogen (Carlsbad, Calif.), Ademtech (Pessac, France), and Miltenyi Biotec GmbH (Bergisch Gladbach, Germany).

The analyte-binding material preferably includes magnetic particulate material wherein each particle of the particulate material has at least two antibodies that bind different analytes disposed thereon. For example, in certain embodiments, the analyte-binding material includes magnetic particulate material having antibodies MAb-107 and affinity-purified GxClf40 disposed thereon (preferably, in a ratio of 1:1).

Antibodies can be attached to magnetic particulate support material through either covalent attachment or non-covalent attachment.

Non-covalent attachment of an antibody to a magnetic particulate support material includes attachment by ionic interaction or hydrogen bonding, for example. One example of a non-covalent attachment included in the present invention is the well-know biotin-avidin system. Avidin-biotin affinity-based technology has found wide applicability in numerous fields of biology and biotechnology. The affinity constant between avidin and biotin is remarkably high (the dissociation constant, Kd, is approximately 10⁻¹⁵ M, see, Green, Biochem. J., 89, 599 (1963)) and is not significantly lessened when biotin is coupled to a wide variety of biomolecules. Numerous chemistries have been identified for coupling biomolecules to biotin with minimal or negligible loss in the activity or other desired characteristics of the biomolecule. A review of the biotin-avidin technology can be found in Applications of Avidin-Biotin Technology to Affinity-Based Separation, Bayer, et al., J. of Chromatography, pgs. 3-11 (1990).

Streptavidin, and its functional homolog avidin, are tetrameric proteins, having four identical subunits. Streptavidin is secreted by the actinobacterium, Streptomyces avidinii. A monomer of streptavidin or avidin contains one high-affinity binding site for the water-soluble vitamin biotin and a streptavidin or avidin tetramer binds four biotin molecules.

Biotin, also known as vitamin H or cis-hexahydro-2-oxo-1H-thieno-[3-4]-imidazole-4-pentanoic acid, is a basic vitamin which is essential for most organisms including bacteria and yeast. Biotin has a molecular weight of 244 daltons, much lower than its binding partners avidin and streptavidin. Biotin is also an enzyme cofactor of pyruvate carboxylase, trans-carboxylase, acetyl-CoA-carboxylase and beta-methylcrotonyl-CoA carboxylase which together carboxylate a wide variety of substrates.

Both streptavidin and avidin exhibit extremely tight and highly specific binding to biotin which is one of the strongest known non-covalent interactions between proteins and ligands, with a molar dissociation constant of 10⁻¹⁵ molar (M) (Green, Advances in Protein Chemistry, Vol. 29, pp. 85-133 (1975)), and a t½ of ligand dissociation of 89 days (Green, N. M., Advances in Protein Chemistry, Vol. 29, pp. 85-133 (1975)). The avidin-biotin bond is stable in serum and in the circulation (Wei et al., Experientia, Vol. 27, pp. 366-368 (1971)). Once formed, the avidin-biotin complex is unaffected by most extremes of pH, organic solvents, and denaturing conditions. Separation of streptavidin from biotin requires conditions, such as 8 M guanidine, pH 1.5, or autoclaving at 121° C. for 10 minutes (min).

Antibodies may be biotinylated using a wide variety of known methodologies. For example, antibodies may be biotinylated chemically, using activated biotin analogues, such as N-hydroxysuccinimidobiotin (NHS-biotin), which is commercially available from Pierce Chemical Company, Rockford, Ill., and requires the presence of a free primary amino group on the antibody.

In a preferred method of the present invention, magnetic particles can be coated with streptavidin and contacted with biotinylated antibodies. These particles can then be used for bacterial capture. With two or more antibodies, simultaneous or sequential capture can occur. Another option is that the biotinylated antibodies may be mixed with the sample to capture the bacteria and the antibody-bacteria complex can then be captured on the bead (Dynal T1 MyOne Streptavidin Package insert).

For certain embodiments, the ratio of the number of biotin molecules to the number of antibodies can be optimized to avoid aggregation for certain particles. For example, with the Ademtech 200-nm streptavidin-coated particles, a ratio of around 2:1 is preferred. Higher ratios, especially greater than 7:1 have shown aggregation issues for these particles.

Representative methods for covalent attaching an antibody to a particulate support material include utilizing functional groups in the support materials (such as carboxyl, amine, hydroxyl, maleimide, hydrazide) activated by activation compounds (such as glutaraldehyde, carbodiimide, cyanogen bromide) to react with another reactive groups (such as hydroxyl, amino, amido, or sulfhydryl groups) in an antibody. This bond may be, for example, a disulfide bond, thioester bond, amide bond, thioether bond, and the like. Antibodies may also be directly attached to support material functionalized with groups (such as tosyl, chloromethyl) that can directly react with a functional group on the antibody (such as amine).

Antibodies may be covalently bonded to magnetic particulate support material by a wide variety of methods known in the art. For example, beads are commercially available that are derivatized with carboxyl groups. Antibodies can then be coupled to these beads through the formation of an amide linkage between a primary amine on the antibody and the carboxyl groups on the bead surface that is mediated by carbodiimide activation.

Typically, the particle concentration and antibody-to-particle ratios are optimized for the system of interest to achieve rapid capture. Generally, this is particle dependent. For example, for Dynal 1-μm particles the particle concentration is preferably greater than 0.04 mg/mL, more preferably greater than 0.1 mg/mL, and even more preferably greater than 0.16 mg/mL. For the same particles, the antibody to particle ratio is preferably greater than 1 μg/mg particles, more preferably greater than 10 μg/mL, and even more preferably greater than 40 μg/mg particles.

For example, for Ademtech 200-nm particles, the particle concentration is preferably greater than 0.04 mg/mL, more preferably greater than 0.1 mg/mL, and even more preferably greater than 0.16 mg/mL. For the same particles, the antibody to particle ratio is preferably at least 0.01 μg/mg particles, more preferably greater than 0.1 μg/mL, and even more preferably greater than 1 μg/mg particles. For the same particles, the antibody to particle ratio is preferably less than 10 μg/mg particles.

Suitable particles may or may not be blocked to prevent nonspecific binding. Such blocking may be done before or after antibody attachment. For example, certain magnetic beads (e.g., Dynal T1 MyOne streptavidin beads) are purchased blocked with bovine serum albumin (BSA). Other suitable blocking agents for nonspecific binding may be used, as is well known in the art.

Contact times (for example, mixing times) between the sample containing the target whole cells and the solid support material containing the antibodies can be no greater than 15 minutes, however as low as 30 seconds and as high as 30 minutes may be used. Such compositions may also include a buffer, such as PBS optionally with a PLURONIC L-64 surfactant, ethylenediamine tetraacetic acid (EDTA), BSA, or a combination thereof. Although physical agitation (or mixing) can be used for both large and small particles, the small particles may be used without mixing.

Particles may be separated from the sample by settling, centrifugation, or filtration. Preferably, magnetic particles are used and they are separated by the use of a magnetic field. Such separated particles (having whole cells thereon) can be washed with various buffers including, for example, PBS with PLURONIC L-64, or TWEEN 20, with or without BSA, etc.

Significantly, using whole cell capture methods of the present invention, preferably at least 20% of the target whole cells in a sample are captured, more preferably at least 50% of the target whole cells are captured, and even more preferably at least 80% of the target whole cells are captured. Methods of the present invention include lysing the target whole cells in the test sample.

The target whole cells may be removed from the magnetic particles prior to lysing. There are both chemical and physical methods for removing cells from the magnetic particles. The simplest methods rely on a change of buffer pH or ionic strength (or both) to release captured cells. Temperature may also be used as a trigger to release captured cells. Another method would rely on providing a labile linking group between the solid support surface and the captured antibody. Depending on the constitution of the linker several modes can be used to trigger the labile component. Light and heat exposures are possible means of triggering a labile linker to release the captured antibody. It would also be possible to release the captured cells by displacing them using a competitive binding agent that has higher affinity for the captured antibody. Alternatively, the target whole cells may be lysed while they are attached to the magnetic particles.

To effectively measure the ATP in cells it is desirable to extract it efficiently without degradation. The literature describes many different extracting treatments. See, for example, Karl D. M.; Microbiology Review, 44, 739, 1980; Stanley, P. E. Methods in Enzymology, 133, 14, 1986. One of the most commonly used extractants is trichloroacetic acid (TCA). TCA is used to release ATP from cells as well as to inactivate enzymes present in a sample matrix that could lead to ATP degradation. Usual TCA concentrations are in the range from 0.5% to 2.5% (by volume), but because TCA inhibits the Luciferin/Luciferase reaction, typically one employs the lowest needed concentration of TCA that can still efficiently extract the ATP content of a given cell target. After extraction, samples are typically neutralized by addition of an appropriate buffer (e.g., Tris-Acetate), to achieve a solution pH of approximately 7.7.

Extraction of ATP through cell lysis can be accomplished in other ways as well. For example, lysing can be conducted under conventional conditions, such as, for example, at a temperature of 5° C. to 42° C. (probably as high as 50° C.), preferably at a temperature of 15° C. to 25° C. Significantly, the lysing can occur using uncultured cells, i.e., a direct test sample, although cultured cells can be used as well.

Lysing can occur upon physically lysing the cells. Physical lysing can occur upon vortexing the test sample with glass beads, sonicating, heating and boiling, or subjecting the test sample to high pressure, such as occurs upon using a French press, for example

Lysing can also occur using a lysing agent. Suitable lysing agents include, for example, enzymes (e.g., protease, glycosidases, nucleases). Exemplary enzymes include lysostaphin, pepsin, glucosidase, galactosidase, lysozyme, achromopeptidase, endopeptidases, N-acetylmuramyl-L-alanine amidase, endo-beta-N-acethylglucosaminidase, ALE-1, DNase, and RNase. Various combinations of enzymes can be used if desired. Lysostaphin is particularly useful in methods of detecting the presence of Staphylococcus aureus.

Other lysing agents include salts (e.g., chaotrophic salts), solubilizing agents (e.g., detergents), reducing agents (e.g., beta-mercaptoethanol (BME), dithiothreitol (DTT), dithioerythritol (DTE), tris(2-carboxyethyl) phosphine hydrochloride (TCEP; Pierce Chemical Company, Rockford, Ill.), cysteine, n-acetyl cysteine), acids (e.g., HCl), and bases (e.g., NaOH). Such lysing agents may be more suitable for certain organisms than for others, for example, they can be more suitable for use with Gram negative bacteria than with Gram positive bacteria.

Various combinations of lysing agents and/or methods can be used if desired. Methods of lysing are further discussed in U.S. Patent Application Publication No. 2005/0153370 A1.

Additionally, if desired, and the sample is a mucus-containing sample, it can be further treated, either before or after lysing, with at least one reagent that can include a mucolytic agent. Treatment of mucus-containing samples with mucolytic agents can reduce the interference resulting from the presence of mucus during the analysis.

Examples of mucolytic agents include enzymes (e.g., pepsin, DNases, RNases, glucosidases, galactosidases, glycosidases), salts (e.g., chaotrophic salts), solubilizing agents (e.g., surfactants, detergents), reducing agents (e.g., beta-mercapto ethanol (BME), dithiotreotol (DTT), dithioerythritol (DTE), cysteine, TCEP, n-acetyl cysteine), and acids (e.g., HCl). Various combinations of such mucolytic agents can be used if desired. One of skill in the art will understand that there can be overlap between lysing agents and mucolytic agents; although not all lysing agents will be mucolytic, for example.

In certain embodiments, if the sample is a mucus-containing sample, and the mucolytic agent is a reducing agent, the reducing agent may be acidified (e.g., having a pH of less than 3). Reducing agents can be acidified using a variety of acids, such as inorganic acids (e.g., HCl) or organic acids (e.g., lactic acid, citric acid). Alternatively, if used in sufficiently high concentrations, the pH of the reducing agent does not need to be adjusted with an acid.

Typically, but optionally, after adding a reducing agent, the sample preparation involves inactivating the reducing agent in the composition. This can be done, for example, by providing a competitive substrate (for example, bovine serum albumen for n-acetyl cysteine). Other examples of reagents that inactivate the reducing agent include a diluent including a neutralizing buffer. Representative ingredients for neutralizing buffers can include, for example, buffering agent(s) (e.g., phosphate), salt(s) (e.g., NaCl), protein stabilizer(s) (e.g., BSA, casein, serum) polymer(s), saccharides, and/or detergent(s) or surfactant(s) (e.g., one or more of the following agents listed by tradenames and commonly available sources: NINATE 411 (amine alkylbenzene sulfonate, available from Stepan Co., Northfield, Ill.), ZONYL FSN 100 (Telomer B monoether with polyethylene glycol, available from E.I. DuPont de Nemours Co.), Aerosol OT 100% (sodium dioctylsulfosuccinate, available from American Cyanamide Co.), GEROPON T-77 (sodium N-oleyl-N-methyltaurate, available from Rhodia Novacare), BIO-TERGE AS-40 (sodium olefin (C₁₄-C₁₆)sulfonate, available from Stepan Co.), STANDAPOL ES-1 (sodium polyoxyethylene(1) laurylsulfate, available from Cognis Corp., Ambler, Pa.), TETRONIC 1307 (ethylenediamine alkoxylate block copolymer, available from BASF Corp.), SURFYNOL 465, 485, and 104 PG-50 (all available from Air Products and Chemicals, Inc.), IGEPAL CA210 (octylphenol ethoxylate, available from Stepan Co.), TRITON X-45, X-100, and X-305 (octylphenoxypolyethoxy ethanols, all available from The Dow Chemical Co.), SILWET L-7600 (polydimethylsiloxane methylethoxylate, available from Momentive Performance Materials, Inc., Wilton, Conn.), RHODASURF ON-870 (polyethoxylated(2) oleyl alcohol, available from Rhodia Novacare), CREMOPHOR EL (polyethyoxylated castor oil, available from BASF Corp.), TWEEN 20 and TWEEN 80 (polyoxyethylene sorbitan monolaurate and monooleate, both available from Sigma-Aldrich Corp.), BRIJ 35 (polyoxyethylene(23) dodecyl ether, available from Sigma-Aldrich Corp.), CHEMAL LA-9 (polyoxyethylene(9) lauryl alcohol, available from PCC Chemax, Piedmont, S.C.), PLURONIC L64 (poly(oxyethylene-co-oxypropylene) block copolymer, available from BASF Corp.), SURFACTANT 10G (p-nonylphenoxypoly(glycidol), available from Arch Chemicals Inc., Norwalk, Conn.), SPAN 60 (sorbitan monostearate, available from Sigma-Aldrich Corp.), CREMOPHOR EL (a polyethoxylated castor oil, available from Sigma-Aldrich Corp.)). If desired, the neutralizing buffer can also be used to adjust the pH of the sample.

In addition to, or alternative to, a reducing agent, the sample preparation of a mucus-containing sample can include the use of one or more surfactants or detergents (e.g., subsequently to or concurrently with, the combining of the sample and the enzymatic lysing agent with the mucolytic agent). Suitable surfactants can be nonionic, anionic, cationic, or zwitterionic. Suitable examples include sodium dodecyl sulfate (SDS) and sodium lauryl sulfate (SLS). Various combinations of surfactants can be used, if desired.

Optionally, the sample preparation method can include subsequently inactivating the surfactant. This can be done, for example, by providing a competitive substrate. Other examples of inactivating the surfactant include using reagent neutralizing buffers, such as a buffer that is sufficient to adjust the pH of the mucolytic test sample and surfactant to a pH of at least 5. Preferably, the buffer is sufficient to adjust the pH to no greater than 8.

Furthermore, if one or more of the sample preparation reagents is acidic, the subsequent composition including the analyte of interest is preferably neutralized to a pH of 7 to 7.5 or near 7.2. This can be done, for example, by providing a buffer and/or a diluent.

The present invention provides various methods of analyzing a sample for a bacterium of interest based on analysis of adenosine triphosphate (ATP). This can be done directly or indirectly.

ATP detection can be used as an indicator of bacterial load. In a direct ATP assay, after separating the solid support with bound bacterial cells from the remainder of the sample (which may contain interfering components such as extra-cellular ATP), the cells are lysed (which may be done in the presence of the magnetic particles) and contacted with luciferin and luciferase. The resulting bioluminescence, which is of an intensity proportional to the number of captured bacterial cells, is then detected, and preferably measured, for example, using a luminometer. Such method is described, for example in McElroy, W. D. and Deluca, M. A.; Firefly and bacterial luminescence: Basic science and applications; Journal of Applied Biochemistry, Vol. 5, 197, (1983), and Lundin, A. and Thore, A.; Analytical information obtainable by evaluation of the time course of firefly bioluminescence in the assay of ATP; Analytical Biochemistry, Vol. 66, 47, (1975). Luciferin/luciferase preparations and methods for their use in a direct ATP assay are well known to those skilled in the art and are commercially available (e.g., ENLITEN rLuciferase/Luciferin Reagent available from Promega Corporation, Madison WI, or Clean-Trace or Aqua-Trace available from Biotrace International, Bridgend UK). A typical formulation contains, for example, 0.1 milligram/liter (mg/L) to 10 mg/L luciferase, 15 micromole/liter (μmol/L) to 1000 μmol/L, preferably 15 μmol/L to 100 μmol/L (e.g., 36 μmol/L) D-luciferin, and agents such as MgCl₂ (2.5-25 mmole) EDTA, BSA, and pH 7 buffer (more typically pH 7.8 buffer).

The relation of the amount of ATP to the amount of bacteria and/or intracellular content characteristic thereof is readily performed by use of calibration curves prepared by performing the assay method using known amounts of target bacteria or intracellular material characteristic thereof and estimating the unknown amount by comparison with this. For a preferred method, a calibration curve of light emitted per number of bacteria will be prepared and a reading of light output per unit time from an unknown amount of material in a sample interpreted from that.

The rate of the luciferase reaction governs the intensity and the rate of decay of the luminescent signal. Thus, any environmental variables impacting the rate of the luciferase reaction will also impact the intensity and the stability in time of the luminescent signal. For example, temperature will affect the rate of the enzymatic reaction and the luminescent output of the system. It is desirable to maintain temperature control while running the assay in order to obtain consistent results.

The light output of the reaction will also vary with time, so it is desirable to understand the kinetic properties of the detection system in order to ensure that the luminescent signal is measure while at its peak intensity. Most commercial reagents are formulated to yield a more constant light output for longer times in order to minimize these kinetic effects.

The chemical environment of the luciferase reaction will also have an impact on the luminescence generated. For example, surfactants and solvents contaminating a sample either at the source, or as a result of processing that sample prior to ATP detection, could modify the expected light output. Thus, it is preferred to adopt appropriate controls and understand the potential sources of interferences to the luciferase reaction that may be present in a particular sample type. Along these lines, it is also important to recognize if the sample being analyzed contains contaminating ATP (i.e., non-bacterial ATP). Although the sample preparation described herein would reduce the amount of contaminating ATP, an enzymatic treatment of the sample could also be used. For example, apyrase could be used to degrade ATP that is not contained in a cell, prior to cell capture, to minimize as much as possible the background signal from contaminant ATP.

Mixing is another variable in ATP detection. Optimum performance results from completely mixing reagents with the extracted ATP.

Also, the time needed to extract ATP from the target cell may depend not only on the extractant reagent used but also on the type of target cell. Extraction of ATP from certain bacteria may take longer. Most ATP reagents will inherently present an ATP background, regardless of their preparation method or purity. This background signal can have an impact on the sensitivity achievable, thus it is desirable to use reagents that are extremely clean of background ATP.

The luciferase/luciferin reagent system should also include thermal stabilizers to maximize their shelf-life. Most commercial luciferase/luciferin reagents are formulated to be heat stable.

In an indirect ATP assay, after separating the solid support with bound bacterial cells from the remainder of the sample (which may contain interfering components such as extra-cellular ATP), the cells are lysed (which may be done in the presence of the magnetic particles) and the lysate contacted with a solution containing adenosine diphosphate (ADP) under conditions effective to produce adenosine triphosphate (ATP) by any adenylate kinase present. Such method is described, for example in U.S. Pat. No. 5,798,214. The use of adenylate kinase can also be used to enrich a sample with ATP.

The amount of ADP with which the sample is mixed is preferably sufficient to provide an ADP concentration in the mixture in excess of 0.005 mM, more preferably in excess of 0.01 mM, and most preferably in excess of 0.08 mM. A particularly preferred amount of ADP in the conversion step mixture is about 0.1 mM. This may depend upon the purity of the ADP: high levels of contamination with ATP restrict higher concentrations being used. The ranges that would be practically useful for ADP are from 10 mM to 0.1 μM.

The conditions effective to produce ATP include the presence of magnesium ions at a molar concentration sufficient to allow maximal conversion of ADP to ATP. For the preferred concentrations of ADP set out above, the preferred concentration of magnesium ions in the suspension or solution during conversion of ADP to ATP is 1 mM or more, more preferably 5 mM or more, and most preferably 10 mM or more. The magnesium ions may be provided in the form of any magnesium salt, but preferably as magnesium acetate. The ranges that would be practically useful for Mg²⁻ are from approximately 0.1 mM to approximately 25 mM. The amount of Mg²⁻ present may depend, among other things, on ADP concentration.

As magnesium ions can cause instability in ADP (in terms of allowing contaminating adenylate kinase to prematurely convert it to ATP) it is preferred not to keep them in solution together prior to use, preferably they are brought together just prior to use or in the ADP conversion step. As magnesium ions are required for the activity of adenylate kinase it may be preferred to mix these and the sample together before adding ADP. Where the reagents are to be kept together it is preferred that they are kept in freeze dried form to avoid any unstabilizing effects.

The conditions effective to produce ATP include incubating the lysate with the ADP and the magnesium ions for a time effective to convert ADP to ATP. Conditions and considerations discussed above for the direct ATP assay apply similarly for an indirect ATP assay.

The ATP produced can be detected, and preferably, the amount of ATP produced can be measured, and related to the presence and/or amount of bacteria or intracellular material characteristic thereof. This can be carried out using a luciferase/luciferin reagent to produce light proportional to the amount of ATP produced, and the light detected using a luminometer in the same manner as in a direct ATP assay. Luciferin/luciferase preparations and methods for their use are described above with respect to the direct ATP assay. A preferred embodiment involves the addition of the luciferin/luciferase luminometry reagents to the sample at the beginning of the incubation, preferably as a single reagent with the ADP and magnesium ion source. This embodiment typically uses a luciferase reagent of high purity. In embodiments of the invention where all the reagents are included at the start of the conversion of ADP to ATP in this manner, and/or where luminometer counting is continued after luciferin/luciferase addition where that is a separate step, magnesium may be provided by the luciferin/luciferase reagent. However, due to binding of magnesium ions by luciferase and EDTA, the amount of magnesium ions is typically positively ensured by prior experiment or calculation. It will be realized by those skilled in the art that the optimal amount of magnesium salt to be added to a given ADP sample and luciferin/luciferase mixture will be readily determinable by routine experimentation using a sample containing a known amount of bacteria whereby maximal signals are obtained.

In an exemplary embodiment, bacteria can be captured and isolated using one, and desirably two antibodies, and analyzed. The isolated bacteria may be enriched in a selective enrichment broth and washed to remove unbound materials. Reagents containing a lysing agent, e.g., lysostaphin, and adenosine diphosphate are added to the washed sample. The lysed cells release adenylate kinase which catalyzes the reaction of ADP to ATP. Luciferin and luciferase are then added to the sample and light is emitted in the presence of ATP.

The present invention also provides a kit or system for carrying out the various methods of the present invention. In one particular embodiment, a kit would include, for example: (1) a sample acquisition device (for example, a device described in U.S. Pat. No. 5,879,635); (2) magnetic beads; (3) a magnet to separate the beads from the sample; (4) sample preparation solutions (e.g., a wash solution for removing non-specifically adsorbed cells and other interfering substances, a lysing agent/extractant to extract the ATP) in appropriate pipettes or reagent bottles, for example, for delivery thereof; (5) Luciferase/Luciferin reagents in appropriate pipettes or reagent bottles, for example, for delivery thereof; and (6) a luminometer to read the light output.

The present invention provides the following illustrative embodiments:
1. A method of analyzing a sample for a bacterium, the method comprising:

providing a sample suspected of including target whole cells comprising one or more analytes characteristic of a specific bacterium;

providing one or more antibodies having antigenic specificities for one or more distinct analytes characteristic of the specific bacterium, wherein the antibodies are selected from the group consisting of MAb-76, MAb-107, affinity-purified RxClf40, affinity-purified GxClf40, MAb 12-9, fragments thereof, and combinations thereof;

providing a solid support material comprising magnetic particles;

providing contact between the sample, the solid support material, and the one or more antibodies under conditions effective to capture target whole cells with one or more analytes characteristic of a specific bacterium, if present;

separating the captured target whole cells from the sample;

lysing the target whole cells to form a lysate; and

analyzing for the presence or absence of the specific bacterium by analyzing the lysate for ATP directly or indirectly.
2. The method of embodiment 1, wherein the one or more antibodies are attached to the solid support material forming an analyte-binding material, and the method includes

providing contact between the sample and the analyte-binding material under conditions effective to capture whole cells with one or more analytes characteristic of a specific bacterium, if present.
3. The method of embodiment 2, wherein providing contact between the sample and the analyte-binding material comprises simultaneous contact between the sample and the one or more antibodies.
4. The method of embodiment 1, wherein providing contact between the sample, the solid support material, and the one or more antibodies comprises providing contact between the one or more antibodies and the sample to form antibody-bound whole cells, and subsequently providing contact between the antibody-bound whole cells and the solid support material.
5. The method of any one of embodiments 1 through 4, wherein the specific bacterium comprises a Gram positive bacterium.
6. The method of embodiment 5, wherein the specific bacterium comprises Staphylococcus aureus.
7. The method of any one of embodiments 1 through 6, wherein at least 20% of the target whole cells are captured.
8. The method of embodiment 7, wherein at least 50% of the target whole cells are captured.
9. The method of embodiment 8, wherein at least 80% of the target whole cells are captured.
10. The method of any one of embodiments 1 through 9, wherein the solid support material comprises particles at a concentration of greater than 0.04 mg/mL.
11. The method of any one of embodiments 1 through 10, wherein the solid support material comprises particles and the antibody to particle ratio is greater than 1 μg/mg particles.
12. The method of any one of embodiments 1 through 11, wherein the solid support material comprises particles and the antibody to particle ratio is at least 0.01 μg/mg particles.
13. The method of embodiment 12, wherein the antibody to particle ratio is less than 10 μg/mg particles.
14. The method of any one of embodiments 1 through 13, wherein each particle has at least two antibodies that bind different analytes disposed thereon.
15. The method of any one of embodiments 1 through 14, wherein the target whole cells are removed from the magnetic particles prior to lysing.
16. The method of any one of embodiments 1 through 15 wherein analyzing for the presence or absence of the specific bacterium by analyzing for ATP directly or indirectly. comprises:

contacting the lysate with a solution containing adenosine diphosphate (ADP) under conditions effective to produce adenosine triphosphate (ATP) by any adenylate kinase present; and

detecting for the presence or absence of produced ATP.
17. The method of embodiment 16 wherein detecting for the presence or absence of produced ATP comprises measuring the amount of adenosine triphosphate (ATP) produced and relating that to the presence and/or amount of specific bacterium or intracellular material characteristic of the specific bacterium.
18. The method of embodiment 16 or embodiment 17 wherein detecting for the presence or absence of produced ATP comprises contacting the mixture containing the lysate and ADP with a luciferase/luciferin reagent to produce light proportional to the amount of ATP produced, and detecting the light with a luminometer.
19. The method of any one of embodiments 16 through 18, wherein the conditions effective to produce ATP include the presence of magnesium ions at a molar concentration sufficient to allow maximal conversion of ADP to ATP.
20. The method of embodiment 19, the conditions effective to produce ATP include incubating the lysate with the ADP and the magnesium ions for a time effective to convert ADP to ATP.
21. The method of any one of embodiments 1 through 15 wherein analyzing for the presence or absence of the specific bacterium by analyzing for ATP directly or indirectly comprises:

contacting the lysate with a luciferase/luciferin reagent to produce light proportional to the amount of ATP present; and

detecting the light using a luminometer.
22. The method of embodiment 21 wherein analyzing for the presence or absence of the specific bacterium further comprises measuring the amount of ATP present and relating that to the amount of bacteria or intracellular material present.
23. A method of analyzing a sample for a bacterium, the method comprising:

providing a sample suspected of including target whole cells comprising one or more analytes characteristic of a specific bacterium;

providing a solid support material comprising magnetic particles having attached thereto one or more antibodies having antigenic specificities for one or more distinct analytes characteristic of the specific bacterium, wherein the antibodies are selected from the group consisting of MAb-76, MAb-107, affinity-purified RxClf40, affinity-purified GxClf40, MAb 12-9, fragments thereof, and combinations thereof;

providing contact between the sample and the magnetic particles having the one or more antibodies attached thereto under conditions effective to capture target whole cells with one or more analytes characteristic of a specific bacterium, if present;

separating the captured target whole cells from the sample; lysing the target whole cells to form a lysate and release adenosine triphosphate (ATP) if present;

contacting the lysate with a luciferase/luciferin reagent to produce light proportional to the amount of ATP present;

detecting the light using a luminometer; and

measuring the amount of ATP present and relating that to the amount of the specific bacterium or intracellular material present characteristic of the specific bacterium.
24. A method of analyzing a sample for a bacterium, the method comprising:

providing a sample suspected of including target whole cells comprising one or more analytes characteristic of a specific bacterium;

providing a solid support material comprising magnetic particles having attached thereto one or more antibodies having antigenic specificities for one or more distinct analytes characteristic of the specific bacterium, wherein the antibodies are selected from the group consisting of MAb-76, MAb-107, affinity-purified RxClf40, affinity-purified GxClf40, MAb 12-9, fragments thereof, and combinations thereof;

providing contact between the sample and the magnetic particles having the one or more antibodies attached thereto under conditions effective to capture target whole cells with one or more analytes characteristic of a specific bacterium, if present;

separating the captured target whole cells from the sample;

lysing the target whole cells to form a lysate;

contacting the lysate with a solution containing adenosine diphosphate (ADP) under conditions effective to produce adenosine triphosphate (ATP) by any adenylate kinase present; andmeasuring the amount of adenosine triphosphate produced and relating that to the amount of the specific bacterium or intracellular material characteristic of the specific bacterium.
25. The method of any one of embodiments 1 through 24 wherein the solid support material comprises magnetic particles having attached thereto two or more antibodies having antigenic specificities for two or more distinct analytes characteristic of the specific bacterium,

### EXAMPLES

The present invention has now been described with reference to several specific embodiments foreseen by the inventor for which enabling descriptions are available. Insubstantial modifications of the invention, including modifications not presently foreseen, may nonetheless constitute equivalents thereto. Thus, the scope of the present invention should not be limited by the details and structures described herein, but rather solely by the following claims, and equivalents thereto.

All parts, percentages, ratios, etc. in the examples and the rest of the specification are by mole unless indicated otherwise. All solvents and reagents without a named supplier were purchased from Aldrich Chemical; Milwaukee, Wis. Water was purified by the use of a U-V Milli-Q water purifier with a resistivity of 18.2 Mohms/cm (Millipore, Bedford Mass.).

Table of Abbreviations

| Abbreviation or Trade Name | Description |
|---|---|
| ATCC | American Type Culture Collection |
| ATP | Adenosine triphosphate |
| PBS buffer | A phosphate buffer saline (PBS) |
| | solution prepared by diluting ten-fold |
| | a 10x PBS liquid concentrate available |
| | commercially from EMD Biosciences, |
| | San Diego, CA |
| PBS L64 buffer | Prepared by taking the PBS buffer |
| | solution and adding 0.2% (w/v) of |
| | PLURONIC L64 |
| PLURONIC L64 | Trade designation for surfactant |
| | available from BASF Corporation, |
| | Mount Olive, NJ |

### Preparative Example 1

### Preparation of Antibody Functionalized Magnetic Beads

Murine anti-Protein A monoclonal antibody, MAb-107, is described in U.S. patent application Ser. No. 11/562,747, filed on Nov. 22, 2006, and PCT Publication No. WO 2008/143697, both entitled “ANTIBODY WITH PROTEIN A SELECTIVITY.” Murine anti-Protein A monoclonal antibody, MAb-107 were biotinylated with EZ-Link NHS-PEO4-Biotin (Product Number 21330) from Pierce according to the manufacturer's directions. Streptavidin-coated magnetic particles (1 μm Dynal T1) were obtained from Invitrogen, Inc. (Carlsbad, Calif.). All reactions and washes were performed in PBS L-64 buffer (phosphate buffered saline with 0.2% w/v PLURONIC L64) unless stated otherwise. Wash steps included three successive washes unless stated otherwise. The washing process consisted of placing a magnet adjacent to the tube to draw the particles to the side of the tube proximal to the magnet, removing the liquid from the tube with the adjacent magnet, and adding an equal volume of fresh buffer to replace the liquid that was removed. The magnet was removed to allow resuspension and mixing the particles.

Streptavidin-coated magnetic particles, at a concentration of 2.5 milligram per milliliter (mg/mL) were mixed with biotinylated antibody preparations in 500 microliters (μL) PBS L-64 buffer. The mass ratio of the antibody to the particles for conjugation was 40 μg antibody/mg of particles. The resulting mixture was incubated at 37° C. for 1 hour (hr). Subsequently, the particles were washed in PBS L-64 buffer to remove unbound antibody. After the final wash the particles were resuspended to a particle concentration of 2.5 mg/mL.

### Preparative Example 2

### Preparation of the Phosphate Buffer Saline With PLURONIC L64 Buffer (PBS-L64 Buffer)

A phosphate buffer saline (PBS) solution was prepared by diluting ten-fold a 10×PBS liquid concentrate (available commercially from EMD Biosciences, San Diego Calif.). This results in a PBS buffer solution with the following salt composition: 10 mM Sodium Phosphate, 137 mM Sodium Chloride, 2.7 mM Potassium Chloride. The PBS buffer solution has a pH of 7.5 at 25° C. To prepare the Phospate Buffer Saline with PLURONIC L64 solution (PBS-L64 buffer solution), 0.2% (w/v) of the PLURONIC L64 surfactant (available from BASF Corporation, Mount Olive, N.J.) was added to the PBS buffer solution. The PBS-L64 buffer solution has a pH of 7.5 at 25° C.

### Preparative Example 3

### Preparation of S. aureus Bacterial Suspension

S. aureus bacteria were obtained from The American Type Culture Collection (Rockville, Md.), under the trade designation ATCC 25923. The bacteria were grown in overnight (17-22 hours at 37° C.) broth cultures prepared by inoculating 5-10 milliliters of prepared, sterile Tryptic Soy Broth (Hardy Diagnostics, Santa Maria, Calif.) with the bacteria. Cultures were washed by centrifugation (8,000-10,000 rpm) for 15 minutes in an Eppendorf model number 5804R centrifuge (Brinkman Instruments, Westbury, N.Y.) and resuspended into PBS L64 buffer and washed by centrifugation for 3 additional cycles with this solution.

### Preparative Example 4

### Preparation of S. epidermidis Bacterial Suspension

S. epidermidis bacteria were obtained from The American Type Culture Collection (Rockville, Md.), under the trade designation ATCC 12228. The bacteria were grown in overnight (17-22 hours at 37° C.) broth cultures prepared by inoculating 5-10 milliliters of prepared, sterile Tryptic Soy Broth (Hardy Diagnostics, Santa Maria, Calif.) with the bacteria. Cultures were washed by centrifugation (8,000-10,000 rpm for 15 minutes in an Eppendorf model number 5804R centrifuge (Brinkman Instruments, Westbury, N.Y.) and resuspended into PBS L64 buffer and washed by centrifugation for 3 additional cycles with this solution.

### Preparative Example 5

### Preparation of the Lysing Solution

A lysing buffer solution was prepared by dissolving under agitation 100 μg of lysostaphin (catalog number L-4402, Sigma-Aldrich) in 10 mL PBS L64 buffer to yield a lysing solution with a lysostaphin concentration of 10 μg/mL.

### Examples 1-6

### Sample To Answer Detection of S aureus

The assay to detect S. aureus was conducted as follows:
(1) Sixteen (16) μL of the magnetic bead suspension functionalized with MAb 107 antibody (as prepared in Preparative Example 1) were added to a polypropylene microcentrifuge tube (available from VWR Scientific, West Chester Pa.). To the same microcentrifuge tube were added 484 μL of the S. aureus bacterial suspension in PBS-L64 buffer (as prepared in Preparative Examples 2 and 3) at a given concentration (reported in Table 1). The mixture was incubated, under rocking agitation using a Barnstead LabQuake shaker (available from Barnstead International, Dubuque Iowa), for 15 minutes at room temperature.
(2) The beads were then separated and concentrated by placing the microcentrifuge tube in a Dynal magnetic fixture (available from Invitrogen, Inc. Carlsbad, Calif.) for at least 5 minutes. The supernatant was discarded by micropipetting without disrupting the agglomerated beads.
(3) The beads were then washed by adding 0.5 mL PBS-L64 buffer to the tube and agitating using a rocking motion for 5 minutes. The beads were then again separated and concentrated by placing the microcentrifuge tube in a Dynal magnetic fixture for at least 5 minutes. The wash solution was discarded by micropipetting without disrupting the agglomerated beads. This wash step was repeated a second time.
(4) One hundred (100) μL of the lysostaphin solution (as prepared in Preparative Example 5) was added to the washed magnetic beads. The microcentrifuge tube was then vortexed for 10 seconds and allowed to stand for 5 minutes.
(5) The beads were then again separated and concentrated by placing the microcentrifuge tube in a Dynal magnetic fixture for at least 5 minutes. All of the supernatant (100 μL) was then micropipetted to the bottom chamber of a Biotrace Aqua-Trace test device after removal of the swab and of the foil sealed chamber containing the lysing agents in pelletized form. The bottom chamber of the Biotrace device contains all the necessary dry reagents (Luciferase/Luciferin and stabilizers) to determine the presence of ATP via luciferase bioluminescence. The sample was vortex for 10 seconds and placed in the Biotrace luminometer (Uni-Lite NG) within thirty seconds after adding the supernatant from step (4) to the biotrace device. The bioluminescent response from each sample is reported in Table 1 in Relative Light Units (RLUs). The average RLUs from three replicates is reported in Table 1. Also shown in Table 1 are the 1σ standard deviation on the reported RLUs values. The table shows the results from two trials which used two different preparations of the same functionalized magnetic beads. In Examples 1-3 a bead preparation was used that had been stored at 4° C. for five months, while Examples 4-6 show the results from using a freshly prepared solution of beads. There was a statistically significant difference between the two trials only at the highest concentration of S. aureus tested. This indicates that the freshly made bead solution was more efficient at capturing the bacteria than the one stored for five months. Independent verification of the capture efficiency of the two batches of bead solutions by plating and culturing the beads after capture, showed that the capture efficiency of the freshly prepared solution was approximately 80% of the total bacterial concentration (10⁶ cfu/ml), while the stored solution had approximately 60% capture efficiency. These examples demonstrate the ability to detect a target organism over a range of concentrations, with a limit of detection as low as 10000 cfu/mL.

TABLE 1

| | S. aureus | | 1σ |
|---|---|---|---|
| | Concentration | Relative Light | standard |
| Example | (cfu/mL) | Units (RLU) | deviation |
| 1 | 0 | 4 | 1 |
| 2 | 14500 | 16.3 | 0.6 |
| 3 | 1450000 | 1166 | 90 |
| 4 | 0 | 3 | 0 |
| 5 | 14500 | 16.7 | 1.2 |
| 6 | 1450000 | 1516 | 70 |

### Examples 7-10

### Sample To Answer Detection of S aureus In the Presence of S. epidermidis

The assay to detect S. aureus in the presence of S. epidermidis was conducted as described for Examples 1-6 with the inclusion of a sample were S. epidermidis (as prepared in Preparative Example 4) was mixed into the solution containing S. aureus in order to demonstrate the detection of a target analyte (S. aureus) in the presence of a significant concentration of an interfering organism (S. epidermidis). The average RLUs from three replicates is reported in Table 2. Also shown in Table 2 are the 1σ standard deviations on the reported RLUs values. Example 7 is the negative control, Examples 8 and 9 are the positive controls for S. aureus and S. epidermidis respectively, and Example 10 is the mixed sample.

TABLE 2

| | S. | S. | | |
|---|---|---|---|---|
| | aureus | epidermidis | | 1σ |
| | Conc. | Conc. | Relative Light | standard |
| Example | (cfu/mL) | (cfu/mL) | Units (RLU) | deviation |
| 7 | 0 | 0 | 4 | 1 |
| 8 | 1350000 | 0 | 759 | 27 |
| 9 | 0 | 1000000 | 81 | 15 |
| 10 | 1350000 | 1000000 | 792 | 39 |

### Examples 11-24

### Effect of Lysing Agent On ATP Detection of Different Bacterial Targets

These examples demonstrate the impact of different lysing agents on the ATP based detection of different bacteria. In Examples 11-17, 10 μL of either a S. aureus bacterial suspension in PBS-L64 buffer (as prepared in Preparative Examples 2 and 3) or a S. epidermidis bacterial suspension in PBS-L64 buffer (as prepared in Preparative Examples 2 and 4) were added to 90 μL of the lysostaphin solution (as prepared in Preparative Example 5) to yield the bacterial concentrations reported in Table 3. Each sample was vortexed for 5 minutes and added to the bottom chamber of a Biotrace Aqua-Trace test device as described in step (5) of the procedure used for Examples 1-6 above. Alternatively, for Examples 18-24, 10 μL of either a S. aureus bacterial suspension in PBS-L64 buffer or a S. epidermidis bacterial suspension in PBS-L64 buffer were spiked onto the swab of a Biotrace Clean-Trace test. The remainder of the test was then carried out in accordance with the manufacturer's instructions. The Clean-Trace test uses non-enzymatic lysing agents. The average RLUs from two replicates is reported in Table 3. Also shown in Table 3 are the 1σ standard deviations on the reported RLUs values. Referring to the table, no significant differences were observed between the detection of S. aureus and S. epidermidis when using non-specific lysing agents such as those employed by the Clean-Trace product. However, a specific enzymatic lysing agent such as lysostaphin can exhibit a differential lysing action useful in the specific detection of a target organism in the presence of closely related bacteria. The combination of a specific sample preparation with a specific lysing step and ATP detection, yields a superior system capable of deteting bacterial concentrations as low as 1000 cfu/mL.

TABLE 3

| | | | S. | Relative | |
|---|---|---|---|---|---|
| | | S. aureus | epidermidis | Light | 1σ |
| | Lysing | Conc. | Conc. | Units | standard |
| Example | Agent | (cfu/mL) | (cfu/mL) | (RLU) | deviation |
| 11 | Lysostaphin | 0 | 0 | 2.5 | 0.7 |
| 12 | Lysostaphin | 1000 | 0 | 7 | 0 |
| 13 | Lysostaphin | 10000 | 0 | 38.5 | 0.7 |
| 14 | Lysostaphin | 1000000 | 0 | 4620 | 669 |
| 15 | Lysostaphin | 0 | 1000 | 2.5 | 0.7 |
| 16 | Lysostaphin | 0 | 10000 | 5 | 1.4 |
| 17 | Lysostaphin | 0 | 1000000 | 187 | 19.8 |
| 18 | Clean-Trace | 0 | 0 | 9.5 | 0.7 |
| 19 | Clean-Trace | 1000 | 0 | 19 | 1.4 |
| 20 | Clean-Trace | 10000 | 0 | 55 | 0 |
| 21 | Clean-Trace | 1000000 | 0 | 4772 | 473 |
| 22 | Clean-Trace | 0 | 1000 | 16.5 | 0.7 |
| 23 | Clean-Trace | 0 | 10000 | 82.5 | 6.4 |
| 24 | Clean-Trace | 0 | 1000000 | 6207 | 127 |

The complete disclosures of all patents, patent applications, publications, and nucleic acid and protein database entries, including for example GenBank accession numbers, that are cited herein are hereby incorporated by reference as if individually incorporated. Various modifications and alterations of this invention will become apparent to those skilled in the art without departing from the scope and spirit of this invention, and it should be understood that this invention is not to be unduly limited to the illustrative embodiments set forth herein.

1. A method of analyzing a sample for a bacterium, the method comprising:
providing a sample suspected of including target whole cells comprising one or more analytes characteristic of a specific bacterium;
providing one or more antibodies having antigenic specificities for one or more distinct analytes characteristic of the specific bacterium, wherein the antibodies are selected from the group consisting of MAb-76, MAb-107, affinity-purified RxClf40, affinity-purified GxClf40, MAb 12-9, fragments thereof, and combinations thereof;
providing a solid support material comprising magnetic particles;
providing contact between the sample, the solid support material, and the one or more antibodies under conditions effective to capture target whole cells with one or more analytes characteristic of a specific bacterium, if present;
separating the captured target whole cells from the sample;
lysing the target whole cells to form a lysate; and
analyzing for the presence or absence of the specific bacterium by analyzing the lysate for ATP directly or indirectly.

2. The method of claim 1, wherein the one or more antibodies are attached to the solid support material forming an analyte-binding material, and the method includes
providing contact between the sample and the analyte-binding material under conditions effective to capture whole cells with one or more analytes characteristic of a specific bacterium, if present.

3. The method of claim 2, wherein providing contact between the sample and the analyte-binding material comprises simultaneous contact between the sample and the one or more antibodies.

4. The method of claim 1, wherein providing contact between the sample, the solid support material, and the one or more antibodies comprises providing contact between the one or more antibodies and the sample to form antibody-bound whole cells, and subsequently providing contact between the antibody-bound whole cells and the solid support material.

5. The method of claim 1, wherein the specific bacterium comprises a Gram positive bacterium.

6. The method of claim 5, wherein the specific bacterium comprises Staphylococcus aureus.

7. The method of claim 1, wherein at least 20% of the target whole cells are captured.

8. The method of claim 7, wherein at least 50% of the target whole cells are captured.

9. The method of claim 8, wherein at least 80% of the target whole cells are captured.

10. The method of claim 1, wherein the solid support material comprises particles at a concentration of greater than 0.04 mg/mL.

11. The method of claim 1, wherein the solid support material comprises particles and the antibody to particle ratio is greater than 1 μg/mg particles.

12. The method of claim 1, wherein the solid support material comprises particles and the antibody to particle ratio is at least 0.01 μg/mg particles.

13. The method of claim 12, wherein the antibody to particle ratio is less than 10 μg/mg particles.

14. The method of claim 1, wherein each particle has at least two antibodies that bind different analytes disposed thereon.

15. The method of claim 1, wherein the target whole cells are removed from the magnetic particles prior to lysing.

16. The method of claim 1 wherein analyzing for the presence or absence of the specific bacterium by analyzing for ATP directly or indirectly comprises:
contacting the lysate with a solution containing adenosine diphosphate (ADP) under conditions effective to produce ATP by any adenylate kinase present; and
detecting for the presence or absence of produced ATP.

17. The method of claim 16 wherein detecting for the presence or absence of produced ATP comprises measuring the amount of adenosine triphosphate (ATP) produced and relating that to the presence and/or amount of specific bacterium or intracellular material characteristic of the specific bacterium.

18. The method of claim 16 wherein detecting for the presence or absence of produced ATP comprises contacting the mixture containing the lysate and ADP with a luciferase/luciferin reagent to produce light proportional to the amount of ATP produced, and detecting the light with a luminometer.

19. The method of claim 16, wherein the conditions effective to produce ATP include the presence of magnesium ions at a molar concentration sufficient to allow maximal conversion of ADP to ATP.

20. The method of claim 19, the conditions effective to produce ATP include incubating the lysate with the ADP and the magnesium ions for a time effective to convert ADP to ATP.

21. The method of claim 1 wherein analyzing for the presence or absence of the specific bacterium by analyzing for ATP directly or indirectly comprises:
contacting the lysate with a luciferase/luciferin reagent to produce light proportional to the amount of ATP present; and
detecting the light using a luminometer.

22. The method of claim 21 wherein analyzing for the presence or absence of the specific bacterium further comprises measuring the amount of ATP present and relating that to the amount of the specific bacterium or intracellular material characteristic of the specific bacterium present.

23. A method of analyzing a sample for a bacterium, the method comprising:
providing a sample suspected of including target whole cells comprising one or more analytes characteristic of a specific bacterium;
providing a solid support material comprising magnetic particles having attached thereto one or more antibodies having antigenic specificities for one or more distinct analytes characteristic of the specific bacterium, wherein the antibodies are selected from the group consisting of MAb-76, MAb-107, affinity-purified RxClf40, affinity-purified GxClf40, MAb 12-9, fragments thereof, and combinations thereof;
providing contact between the sample and the magnetic particles having the one or more antibodies attached thereto under conditions effective to capture target whole cells with one or more analytes characteristic of a specific bacterium, if present;
separating the captured target whole cells from the sample;
lysing the target whole cells to form a lysate and release adenosine triphosphate (ATP) if present;
contacting the lysate with a luciferase/luciferin reagent to produce light proportional to the amount of ATP present;
detecting the light using a luminometer; and
measuring the amount of ATP present and relating that to the amount of the specific bacterium or intracellular material present characteristic of the specific bacterium.

24. A method of analyzing a sample for a bacterium, the method comprising:
providing a sample suspected of including target whole cells comprising one or more analytes characteristic of a specific bacterium;
providing a solid support material comprising magnetic particles having attached thereto one or more antibodies having antigenic specificities for one or more distinct analytes characteristic of the specific bacterium, wherein the antibodies are selected from the group consisting of MAb-76, MAb-107, affinity-purified RxClf40, affinity-purified GxClf40, MAb 12-9, fragments thereof, and combinations thereof;
providing contact between the sample and the magnetic particles having the one or more antibodies attached thereto under conditions effective to capture target whole cells with one or more analytes characteristic of a specific bacterium, if present;
separating the captured target whole cells from the sample;
lysing the target whole cells to form a lysate;
contacting the lysate with a solution containing adenosine diphosphate (ADP) under conditions effective to produce adenosine triphosphate (ATP) by any adenylate kinase present; and
measuring the amount of adenosine triphosphate produced and relating that to the amount of the specific bacterium or intracellular material characteristic of the specific bacterium.

25. The method of claim 1 wherein the solid support material comprises magnetic particles having attached thereto two or more antibodies having antigenic specificities for two or more distinct analytes characteristic of the specific bacterium,
